(19) United States
(12) Patent Application Publication
(10) Pub. No.: US 2015/0050499 A1
(43) Pub. Date: Feb. 19, 2015

(54) FIBROSIS CAUSING AGENT

(71) Applicant: TERUMO KABUSHIKI KAISHA, TOKYO (JP)

(72) Inventors: SUGURU HATA, KANAGAWA (JP); YUICHI TADA, KANAGAWA (JP); AYAKA AKUTAGAWA, KANAGAWA (JP)

(21) Appl. No.: 14/463,470

(22) Filed: Aug. 19, 2014

(30) Foreign Application Priority Data
Aug. 19, 2013 (JP) 2013-169749

(51) Int. Cl. A61K 31/734; A61K 9/14

(52) U.S. Cl. CPC A61K 31/734; A61K 9/14
USPC 428/402; 536/3

(57) ABSTRACT

Disclosed herein is a fibrosis-causing agent in a particulate form which effectively reduces the lung capacity in a noninvasive manner. The particulate form effective in reducing lung capacity in a noninvasive manner remains at or on an affected part of the lung to promote and/or induce fibrosis

### CROSS REFERENCE TO RELATED APPLICATION

This application is based on Japanese Application No. 2013-169749, filed on Aug. 19, 2013, the contents of which are incorporated herein by reference.

### BACKGROUND OF THE DISCLOSURE

The present description relates to a fibrosis-causing agent.

Among a large variety of pulmonary diseases which hamper normal respiration is chronic obstructive pulmonary disease (COPD). It includes at least one of asthma, pulmonary emphysema, and chronic bronchitis, which occludes the lung. These diseases often give rise to their symptoms at one time, thereby making it difficult to determine which one of them causes lung occlusion in each case. COPD remains unchanged for several months and hence chronic bronchitis is clinically identified from the continued reduction of expiration for two or more years. The most serious symptoms relating to COPD are chronic bronchitis and pulmonary emphysema.

The pulmonary emphysema is characterized by an extraordinary expansion, accompanied by disorganization, of respiratory bronchioles, pulmonary alveoli, and alveolar sacs, which are collectively called alveolar parenchyma for gas exchange. The alveolar parenchyma in its normal state shrinks at the time of expiration; however, the enlarged alveolar parenchyma does not recover after expansion due to breathing. This prevents satisfactory expiration. Moreover, the pulmonary emphysema decreases the effective area of pulmonary alveoli and the number of capillary vessels running in all directions on the surface of pulmonary alveoli, which reduces the overall ventilating capacity of the lung. In addition, the lung suffering from pulmonary emphysema is poor in resilience and unable to keep the airway open by stretching because it has its elastin and collagen destroyed by inflammation. This makes the bronchus liable to deformation. The result is that as the lung shrinks for expiration the bronchus becomes narrow due to compression by its surrounding air-filled alveoli and the lung excessively expands, thereby preventing smooth expiration. This is the reason why patients with pulmonary emphysema do expiration while keeping their lips pursed up.

In Japan, there are about 50,000 patients suffering from pulmonary emphysema, who receive home oxygen therapy. Moreover, those who are in the incipient or moderate stage of pulmonary emphysema are estimated to count up to about three millions. The present medical treatment of pulmonary emphysema relies mostly on drug therapy and home oxygen therapy. The oxygen therapy is often applied to those patients who are incapable of absorbing sufficient oxygen from air on account of their severely damaged lung function. It merely alleviates the symptom and is not necessarily wholly effective. The drug therapy is achieved in several ways, such as administration of bronchodilator to open the airway in the lung, thereby alleviating dyspnea; administration of oral or inhalational steroid to alleviate inflammation in the airway; administration of antibiotics to prevent and treat accompanying infection; and administration of expectorant to make the airway free of viscous fluids. Any drug therapy in the foregoing methods helps control and alleviate pulmonary emphysema to some extent but is not highly effective. There are also surgical therapies such as lung implantation and lung contraction (for expansion of normal parts in the lung by removal of damaged parts from the lung). They involve difficulties of securing lungs for implantation and impose a large burden on patients.

More patients will have the chance of receiving treatment if it becomes possible to perform lung volume reduction (LVR) in a noninvasive manner without thoracotomy. Unfortunately, currently available noninvasive surgical therapies are not so successful. One of them reported so far is the injection of a fluid therapeutic agent into the lung or bypass, which promotes fibrosis in lung tissues. (See, for example, U.S. Patent Application Publication No. 2003/0228344.)

### SUMMARY OF THE DISCLOSURE

A drawback of the foregoing fluid therapeutic agent, as mentioned in U.S. Patent Application Publication No. 2003/0228344, is the incapability of stably remaining in affected lung regions (such as pulmonary alveoli and alveolar sacs). This drawback leads to insufficient fibrosis in the target affected part. Although the noninvasive lung volume reduction is strongly required as an effective therapy for pulmonary emphysema, there is actually no satisfactory therapy in this field.

The observations as set forth in the present disclosure have been made in view of the foregoing circumstances, and it is an intention of the present disclosure to provide a fibrosis-causing agent which is effective for lung volume reduction in noninvasive manner.

It is another intention of the present disclosure to provide a method for lung volume reduction in noninvasive manner that employs the foregoing fibrosis-causing agent.

As a result of extensive research, the present inventors found that the foregoing problems are solved by employing a fibrosis-causing agent in particulate form. This finding led to the present disclosure.

In other words, the above-mentioned intention is achieved by the fibrosis-causing agent in particulate form.

The fibrosis-causing agent in particulate form as specified in the present disclosure securely remains on the target affected part, so that it efficiently induces and promotes fibrosis.

### BRIEF DESCRIPTION OF THE FIGURES

FIG. 1 shows optical photomicrographs (×200) of the lung tissues which were given respectively the batch 1 of particles in Example 1, an aqueous solution of sodium alginate in Comparative Example 1, and a gel in Comparative Example 2; and

FIG. 2 shows optical photomicrographs (×400, ×100, and ×200) of the lung tissues which were given respectively the batches 1, 2, and 3 of particles in Example 1.

### DETAILED DESCRIPTION OF THE DISCLOSURE

The present disclosure relates to a fibrosis-causing agent in particulate form (which will be simply referred to as “fibrosis-causing agent” hereinafter). Upon administration to a living organism, the fibrosis-causing agent is recognized as a foreign body which helps grow connective tissues (particularly fibrocytes), thereby inducing and promoting fibrosis. Being in particulate form, the fibrosis-causing agent exhibits low or no fluidity, and hence it hardly or never flows out of the affected part to which it has been administered. Consequently, the fibrosis-causing agent stays on the affected part and efficiently induces and promotes fibrosis. For the fibrosis-causing agent to produce its effect, it is administered to the affected part (such as pulmonary alveoli and alveolar sacs) of patients suffering from pulmonary emphysema, so that it induces and promotes local fibrosis and atrophy in the affected part, thereby reducing the lung capacity. Therefore, the fibrosis-causing agent can be properly used for the therapy of pulmonary emphysema.

The present disclosure will be described in more detail with reference to the following embodiments, which are not intended to restrict the scope thereof.

Symbols and terms used in this specification are defined as follows. The symbol “X to Y” denoting a range implies “no smaller than X and no larger than Y.” The following terms are synonymous with each other: “weight” and “mass”; “wt %” and “mass %”; and “parts by weight” and “parts by mass.” In addition, it is assumed that operations and measurements are carried out at room temperature (20 to 25° C.) and 40 to 50% RH (relative humidity), unless otherwise stated.

Fibrosis-Causing Agent in Particulate Form

The fibrosis-causing agent according to the present disclosure takes on a particulate form. The term “particulate form” means a spherical (or nearly spherical) solid, suggesting that the fibrosis-causing agent substantially lacks fluidity unlike liquid (such as solution, suspension, and emulsion) and gel. The particles may be solid or hollow ones. After the fibrosis-causing agent has been administered to the target affected part, the particles securely stay there, without substantial outflow. The term “substantial” means a relative amount in the total amount, accounting for 50 to 100 wt %, preferably 75 to 100 wt %, more preferably 90 to 100 wt %, and further preferably 95 to 100 wt %.

The fibrosis-causing agent according to the present disclosure is not specifically restricted in the size of particles. To be concrete, the size of particles should have a diameter smaller than twice, preferably lx, the diameter of the entrance of the enlarged pulmonary alveolus or alveolar sac. Particles of this size securely stay on the affected part, without flowing out of it. The entrance diameter of the enlarged pulmonary alveolus or alveolar sac varies depending on the seriousness of pulmonary emphysema, the type and weight of the patient, and the position of the affected part; it is about 1 to 2 mm in the case of human patient. Therefore, the fibrosis-causing agent for human patients of pulmonary emphysema should preferably have a particle diameter no larger than 2 mm. The particle size is not specifically restricted in its lower limit; it should be larger than (preferably 1.1 times) the entrance diameter of the normal pulmonary alveolus or alveolar sac. This particle size is adequate for the fibrosis-causing agent to be selectively introduced and administered to the affected part (enlarged pulmonary alveolus or alveolar sac) without the possibility of entering the normal pulmonary alveolus or alveolar sac. Incidentally, the entrance diameter of the normal pulmonary alveolus or alveolar sac is about 200 to 300 μm in the case of human, although it varies depending on the type and weight of the patient and the position of the affected part.

The term “particle diameter” in this specification means the maximum distance between any two points on the particle contour at which the particle contour crosses the line passing through the particle center. The particle diameter of any particle with an indeterminate form is defined as the maximum length of the particle. The particle diameter may be measured by observation under a scanning electron microscope (SEM), transmission electron microscope (TEM), or optical microscope. It is calculated by averaging particle diameters observed in several to dozens of visual fields. Alternatively, it may be measured by using a particle size distribution measuring apparatus. The particle diameter of the fibrosis-causing agent should preferably be as small as possible from the standpoint of the effect of inducing and promoting fibrosis at the affected part. This is because the fibrosis-causing agent has a larger surface area per unit weight as its diameter decreases. The large surface area leads to a large area of contact with the affected part (enlarged pulmonary alveolus or alveolar sac), which produces the effect of promoting fibrosis. Additional effects include an efficient and easy delivery of the fibrosis-causing agent to the affected part (enlarged pulmonary alveolus or alveolar sac). For the reasons mentioned above, the fibrosis-causing agent of the present disclosure should have a particle diameter no larger than 2000 μm, preferably no larger than 1000 μm, more preferably no larger than 100 μm. The fibrosis-causing agent in fine particle form as mentioned above has a sufficiently large surface area which helps induce and promote the growth of connective tissues (especially fibrocytes) at the contact point upon administration, thereby promoting fibrosis more efficiently. The particle size of the fibrosis-causing agent is not specifically restricted in its lower limit. However, it should have an adequately small size which prevents phagocytosis by macrophages or dendritic cells. Since particles ranging from 200 nm to 5 μm in size are subject to phagocytosis by macrophages, the fibrosis-causing agent should have a particle diameter more than 200 nm, preferably no smaller than 1 μm, and more preferably more than 5 μm. The foregoing size is desirable for the fibrosis to be protected from phagocytosis by macrophages and dendritic cells after administration. Thus, the fibrosis-causing agent is almost entirely introduced and administered to the affected part as intended. Moreover, the fibrosis-causing agent having the particle size specified above effectively prevents inflammation from occurring at the affected part to which it has been administered.

The particles of the fibrosis-causing agent may have its surface modified for protection from phagocytosis by macrophages and dendritic cells. The surface-modified particles may have a smaller size than specified above. However, the minimum size in this case should be 10 nm, which is large enough to prevent inflammation. The above-mentioned surface modification may be supplemented with or replaced by surface treatment with plasma or polyethylene glycol or surface ionization (such as anionization and cationization) for the purpose of enhancement in adhesiveness and/or fibrosis.

The fibrosis-causing agent according to the present disclosure may be formed, without specific restrictions, from or in combination with any material or compound which causes and promotes fibrosis through the growth of connective tissues (particularly fibrocytes) at the affected part. Examples of such materials include biodegradable material, any material that prevents the growth of tissue cells, flexible cured polymer, and adhesive material.

Examples of the biodegradable material or compounds include, but are not limited to, fibrin, fibrinogen, alginate (such as sodium alginate, potassium alginate, ammonium alginate, and calcium alginate), alginic ester, thrombin, borate, calcium, magnesium, chondroitin sulfate, polyamino acid, poly-L-lysine (PLL), poly-L-arginine, poly-ornithine, hyaluronic acid, protein (such as gelatin), starch, collagen, glucosaminoglycan, agarose, dextran, pullulan, heparin, polyglycolic acid, polylactic acid, polyaspartic acid, polycaprolactone, polyhydroxybutyric acid, polydioxanone, “plastarch,” zein, polydioxane, polylactic acid-glycolic acid copolymer, polysaccharide, soybean protein, phospholipid, cholesterol, phospholipid-cholesterol copolymer, polymalic acid, sacran, polyhydroxy butyrate/valerate, polycaprolactone, polybutylene succinate, polybutylene succinate/adipate, polyethylene succinate, aliphatic polyester, vinyl acetate, methyl acrylate, vinyl acetate-methyl acrylate copolymer, biomaterial (such as autologous blood, blood cell component, serum, plasma, bone marrow fluid, fat, and stem cells), and decomposition product resulting from decrosslinking. Additional examples are biodegradable materials disclosed in Japanese Patent Laid-open Nos. 2000-160034 and 2002-146219.

Examples of the material or compounds that prevents the growth of tissue cells include, without specific restrictions, polycationic polymers (such as polycation, composite material of polycation and polyanion, polyvinylamine, polyallylamine), which are disclosed in PCT International Patent Application No. PCT/JP2009/514860.

The polycation may be a polyamino acid or synthetic polypeptide having a plurality of positive charges or net positive charges. Examples of the polyamino acid include poly-D-lysine, poly-L-lysine, poly-DL-lysine, polyarginine, polyhistidine, polyornithine, and polyethylamine. The synthetic polypeptide may be a homopolymer of one kind of positively charged (or basic) amino acid, such as lysine, arginine, and histidine, or a heteropolymer of more than one kind of positively charged amino acid. The foregoing polymer may additionally contain more than one kind of positively charged nonstandard amino acid, such as ornithine and 5-hydroxylysine. The polypeptide may be made functional with such a group as poly(γ-benzyl-L-glutamate). The polycation is not specifically restricted in size; it may be composed of as many amino acid residues as 100 to 4000, 200 to 3000, 300 to 2000, and 500 to 1000. Incidentally, the foregoing polyamino acid and synthetic polypeptide may be produced by any known method, such as chemical synthesis or recombinant DNA technology.

The polycation should be one which has a molecular weight of 10 to 500 kD, preferably 20 to 250 kD, more preferably 50 to 200 kD. This molecular weight may be determined by any known method, such as electrophoresis, size exclusion chromatography, and multiangle laser beam scattering.

The polyanion that forms a composite material with the polycation includes the following without specific restrictions: heparan sulfate, heparin/heparan sulfate, dermatan sulfate, condroitin sulfate, pentosan sulfate, keratan sulfate, keratin sulfate, mucopolysaccharide polysulfate, carrageenan, sodium alginate, potassium alginate, hyaluronic acid, polyglutamic acid, polyaspartic acid, polycarboxymethylcellulose, randomly structured nucleic acid; polysaccharides (such as cellulose, xylose, N-acetyl-lactosamine, glucuronic acid, mannuronic acid, and guluronic acid), sulphated products thereof, and carboxymethylated products thereof; polyamino acid containing a plurality of amino acids selected from the group consisting of Asp, Glu, Lys, Orn, Arg, Gly, Ala, Val, Leu, Ile, Met, Pro, Phe, Trp, Asn, Gln, Ser, Thr, Tyr, Cys, and His, with Asp and/or Glu accounting for no less than about 25% of the amino acids and Lys, Orn, and Arg accounting for no more than about 5% of the amino acids; and polyamino acid represented by any of the formulas poly(X-Y), poly(X-Y-Y), and poly(X-Y-Y-Y), where X independently denotes Asp or Glu, and Y independently denotes Gly, Ala, Val, Leu, Ile, Met, Pro, Phe, Trp, Asn, Gln, Ser, Thr, Tyr, Cys, or His.

A typical example of the flexible cured polymer is polycyanoacrylate formed from cyanoacrylate monomer which polymerizes upon contact with water. Examples of the cyanoacrylate monomer include the following without specific restrictions: alkyl and cycloalkyl α-cyanoacrylate, such as methyl α-cyanoacrylate, ethyl α-cyanoacrylate, propyl α-cyanoacrylate, butyl α-cyanoacrylate, cyclohexyl α-cyanoacrylate, heptyl α-cyanoacrylate, and octyl α-cyanoacrylate; alkenyl and cycloalkenyl α-cyanoacrylate, such as allyl α-cyanoacrylate, methallyl α-cyanoacrylate, and cyclohexenyl α-cyanoacrylate; alkynyl α-cyanoacrylate, such as propangyl α-cyanoacrylate; aryl α-cyanoacrylate, such as phenyl α-cyanoacrylate and toluoyl α-cyanoacrylate; heteroatom-containing methoxyethyl α-cyanoacrylate, ethoxyethyl α-cyanoacrylate, and furfuryl α-cyanoacrylate; and trimethylsilylmethyl α-cyanoacrylate, trimethylsilylethyl α-cyanoacrylate, trimethylsilylpropyl α-cyanoacrylate, and dimethylvinylsilylmethyl α-cyanoacrylate, all of which contain a silicon atom.

Examples of the adhesive material include the following without specific restrictions: talc, tetracycline, Picibanil (OK432), anticancer drug, povidone-iodine, and silver nitrate, which chemically stimulate the pleura, thereby causing pleuritis. The talc is hydrated magnesium silicate [Mg3Si4O10(OH)2], which is composed mainly of SiO2 (about 60%), MgO (about 30%), and water of crystallization (about 4.8%). The Picibanil (OK432) is Streptococcus pyogenes (Group A, Type 3) strain Su (a species of hemolytic streptococcus), in the form of penicillin-treated freeze-dried powder. The anticancer drug includes bleomycin, cisplatin, etc.

The foregoing material may be supplemented with or replaced by any of the following materials, which are disclosed in PCT International Patent Application No. 2009514860: polyvinyl alcohol, gellan gum (which is a polysaccharide resin of high molecular weight obtained from carbohydrate by pure culture fermentation with Pseudomonas elodea and ensuing processes for recovery and purification with isopropyl alcohol, drying, and crushing), gellan gum salt (sodium salt and potassium salt), boronate, poly-ethylamine, polyhistidine, cellulose, xylose, N-acetyllactosamine, glucuronic acid, mannuronic acid, guluronic acid, heparan sulfate, dermatan sulfate, pentosan sulfate, keratan sulfate, mucopolysaccharide polysulfate, carrageenan, carboxymethyl cellulose, hydrogel, acrylamide, agarose, keratin, chitin, chitosan, partially deacetylated chitin, basic polysaccharide (such as aminated cellulose) acrylamide, polyurethane, polyethylene, polyester, fluoroplastics, silica, silicone, hydroxyapatite, ceramics, bone cement, glass, metal, silicon compound, siloxane, crosslinked polymer, porous material, and such material as disclosed in Japanese Patent Laid-open No. 2001-164127.

Preferable among these materials and compounds are: alginates (such as sodium alginate, potassium alginate, ammonium alginate, and calcium alginate), alginic ester, calcium, magnesium, gelatin, collagen, agarose, dextran, polyglycolic acid, polylactic acid, polylactic acid-glycolic acid copolymer, soybean protein, phospholipid, phospholipid-cholesterol polymer, vinyl acetate-methyl acrylate copolymer.

Most desirable among the preferred materials and compounds are: alginate and alginic ester.

The above-mentioned materials for fibrosis may be used alone or in combination with one another.

The fibrosis-causing agent according to the present disclosure should essentially contain any of the foregoing materials for fibrosis. It may additionally contain fat, surface active agent, and adjuvants for its improvement in form stability and functions. The adjuvants may be properly selected, without specific restrictions, according to the type and seriousness of disease. Typical adjuvants include the following: penicillin antibiotics (such as penicillin and viccillin (sodium ampicillin)), aminoglycoside antibiotics, tetracycline, sulfonamide, p-aminobenzoic acid, diaminopyridine, quinolone, β-lactam, β-lactamase inhibitor, chloramphenicol, macrolide, cephalosporin, linomycin, clindamycin, spectinomycin, polymixin B, colistin, vancomycin, bacitracin, isoniazid, rifampicin, ethambutol, ethionamide, aminosalicylic acid, cycloserine, capreomycin, sulphone, clofazimine, thalidomide, polyene antifungal drug, flucytosine, imidazole, triazole, griseofulvin, terconazole, butoconazole, ciclopirox, ciclopirox olamine, haloprogin, tolnaftate, naftifine, terbinafine, and other antiinfectants; radiopaque materials (such as metrizamide, iopamidol, sodium iothalamate, iodamide sodium, and meglumine, which are water-soluble, and gold, titanium, silver, stainless steel, aluminum oxide, and zirconium oxide, which are water-insoluble); contrast enhancer (such as paramagnetic material, heavy atoms, transition metal, lanthanide, actinide, dye, and radioactive nuclear species); steroid; bronchodilator; fibroblast growth factor (FGF), vascular endothelial growth factor (VEGF), nerve growth factor (NGF), epidermal growth factor (EGF), insulin-like growth factor (IGF), transforming growth factor (TGF), brain-derived neurotrophic factor (BDNF), granulocyte colony stimulating factor (G-CSF), granulocyte macrophage colony stimulating factor (GM-CSF), platelet-derived growth factor (PDGF), erythropoietin (EPO), thrombopoietin (TPO), basic fibroblast growth factor (bFGF or FGF2), hepatocyte growth factor (HGF), bone morphogenetic protein (BMP), neurotrophin (neurotrophic factor: NGF, BDNF, NT3, etc.), and other growth factors belonging to the family of the above-mentioned factors;

biomaterial, such as platelet-rich plasma (PRP), autologous blood, serum, plasma, blood cell component, bone marrow fluid, fat, fat stem cells, and mesenchymal stem cells; acylglycerol, neutral fat, wax, ceramide, phospholipid, sphingophospholipid, glycerophospholipid, glycolipid, sphingoglycolipid, glyceroglycolipid, lipoprotein, sulfolipid, isoprenoid, fatty acid, terpenoid, steroid, carotenoid, and other lipids; and

anionic surface active agent (such as sodium salt of fatty acid, monoalkyl sulfate, alkylpolyoxyethylene sulfate, alkylbenzene sulfonate, and monoalkyl phosphate); cationic surface active agent (such as alkyltrimethyl ammonium salt, dialkyldimethyl ammonium salt, and alkylbenzyldimethyl ammonium salt), amphoteric surface active agent (such as alkyldimethylamineoxide and alkylcarboxybetaine), and nonionic surface active agent (such as polyoxyethylene alkyl ether, fatty acid sorbitan ester, alkyl polyglycoside, fatty acid diethanolamide, and alkyl monoglyceryl ether.

The additional adjuvants mentioned above may be used alone or in combination with one another. In the case where the fibrosis-causing agent of the present disclosure contains adjuvants, the amount of the adjuvants may vary, without specific restriction, depending on the type and seriousness of the disease to which it is applied. The content of the adjuvants should preferably be approximately 1 to approximately 200 wt % based on the amount of the fibrosis-causing agent.

The fibrosis-causing agent of the present disclosure may be produced, without specific restrictions, in any known manner as such or with modification. For instance, the process for production from calcium alginate involves spraying one solution to another. The fibrosis-causing agent of the present disclosure should have a prescribed particle size, which is achieved by classification through sieves into fine particles, medium particles (target size particles), and coarse particles. Classification may be accomplished by using any known classifier, such as sieving classifier (for separation of particles by sifting), gravity classifier of horizontal or vertical flow type (for separation of particles by difference in upward or downward flow rates), centrifugal classifier (for separation of particles in the centrifugal force field), and inertia classifier (for separation of particles by abrupt change in the flow direction of air containing particles). Any other methods than mentioned above may also be used with or without modification.

Field of Application

The fibrosis-causing agent of the present disclosure does not flow out of the affected part after administration owing to its form. In other words, the fibrosis-causing agent of the present disclosure efficiently stays at the affected part, thereby properly inducing and promoting fibrosis there. The affected part to which it is applied is not specifically restricted; however, it should preferably be administered to enlarged pulmonary alveoli or alveolar sacs of a patient suffering from pulmonary emphysema, so that it induces and promotes local fibrosis and atrophy in the affected part, thereby reducing the lung capacity. Thus, the fibrosis-causing agent of the present disclosure is applicable to lungs and suitable for therapy of pulmonary emphysema.

The fibrosis-causing agent of the present disclosure may be administered to the affected part of the lung by the method which consists of (a) inserting a catheter into the trachea, bronchus, or bronchiole through the respiratory tract and (b) delivering the fibrosis-causing agent to the respiration region (including pulmonary alveoli or alveolar sacs) through the catheter. This method is intended to promote fibrosis in pulmonary alveoli or alveolar sacs, thereby curing pulmonary emphysema.

The term “respiratory region” used in this specification generically denotes the respiratory organ beyond the bronchus, including respiratory bronchioles and two alveoli. To be concrete, the respiratory region includes bronchi, bronchioles, terminal bronchioles, respiratory bronchioles, alveolar ducts, pulmonary alveoli, alveolar sacs, pulmonary veins, and pulmonary arteries. It should preferably include respiratory bronchioles, alveolar ducts, pulmonary alveoli, alveolar sacs, pulmonary veins. In this specification, the term “pulmonary alveoli or alveolar sacs” denotes at least either of pulmonary alveoli or alveolar sacs, and they are collectively called “alveolar parenchyma.”

The fibrosis-causing agent may be administered by the foregoing method to any object, particularly mammals, without specific restrictions. Typical examples of mammals include human, pet, household animal, and farm animal (such as rabbit, dog, cat, horse, sheep, goat, primate, cow, pig, rat, and mouse). Preferable among them are human, rabbit, dog, and pig, with human being most desirable.

The foregoing method for administration may be applied in the following manner, which is not necessarily restrictive.

Step (a)

This step involves insertion of a catheter into the trachea, bronchus, or bronchiole through the respiratory tract. The catheter may be inserted into any position, however, it should preferably be inserted such that its forward end extends as far as the eighth branch or beyond it. The reason for this is that the opening of enlarged pulmonary alveolus usually exists beyond the eighth to twelfth branches. The catheter inserted in this manner permits (in step (b) that follows) the fibrosis-causing agent to be delivered in a maximum amount selectively to a narrow affected part, i.e., the enlarged pulmonary alveolus or alveolar sac (which are simply referred to as “enlarged alveolar parenchyma” hereinafter). As a result, the thus administered fibrosis-causing agent effectively induces fibrosis. In addition, insertion of the catheter up to or beyond the eighth branch prevents the fibrosis-causing agent from entering the normal pulmonary alveoli or alveolar sacs (which are simply referred to as “normal alveolar parenchyma” hereinafter). This is an effective way of preventing normal pulmonary alveoli or alveolar sacs from fibrosis while keeping them intact. In view of the foregoing, the catheter for treatment of a human patient should preferably be one which has a diameter of 1.5 to 5 mm, more preferably 2 to 4 mm. Incidentally, the first right-left branch of the trachea is defined as the first branch in this specification.

The catheter is not specifically restricted; it may be properly selected according to the diameter (or the number of branches) of the bronchus or bronchiole into which it is inserted. To be concrete, acceptable catheters include any known medical ones for the respiratory organ, circulatory organ, digestive organ, and the catheter disclosed in U.S. Patent Application Publication No. 2006/0283462. Moreover, the catheter is not specifically restricted in its structure; it may or may not have a balloon. The one having a balloon is preferable from the standpoint of easy delivery and administration of the fibrosis-causing agent into the trachea. The catheter is not restricted either in the number of lumens and the inside diameter. Adequate values for them should be selected according to the fibrosis-causing agent to be administered (which varies in number, diameter, and adjuvants) and the presence or absence of a balloon.

Insertion of a catheter into the vicinity of an enlarged alveolar parenchyma may be accomplished with the help of a sheath inserted into a part close to the enlarged alveolar parenchyma. The sheath is not specifically restricted in structure, it may or may not have a balloon. However, it should preferably have a balloon which closes the bronchus or bronchiole. The balloon fixes the sheath to the bronchus or bronchiole, thereby allowing the catheter to be stably inserted into the desired position. The balloon attached to the sheath and the balloon attached to the catheter may be placed at any position in the bronchus or bronchiole without specific restrictions. It is desirable that the balloon attached to the sheath be placed at the bronchus and the balloon attached to the catheter be placed at the bronchus near the terminal, particularly at the bronchiole. Closing the bronchus or bronchiole with a balloon as mentioned above increases airtightness in the region beyond the sheath, thereby allowing the fibrosis-causing agent to be introduced and administered efficiently into the enlarged alveolar parenchyma through the catheter. It is possible to cause two balloons attached to the sheath and catheter respectively to close different parts in the bronchus or bronchiole, so as to easily control the pressure on the normal alveolar parenchyma (existing between the two balloons) or the pressure on the enlarged alveolar parenchyma) beyond the balloon of the catheter.

Closing the bronchus or bronchiole with the balloon of the sheath ensures ventilation with respiration pressure in the near side from the balloon of the sheath. This leads to efficient and safe treatment. The balloon of the sheath may be inflated and deflated in any way without specific restrictions, for example, by means of a three-way stopcock attached to the base of the sheath.

It is possible to stably manipulate the fore-end of the catheter if the pressure is kept constant in the region beyond the balloon attached to the sheath. This is accomplished, for example, by closing the bronchus or bronchiole with the balloon of the sheath and decompressing the region beyond the sheath. This procedure permits the balloon of the catheter to closely adhere to the wall of the bronchus or bronchiole and also prevents air from entering the region beyond the catheter through the side passage. The result is easy decompression in the region beyond the catheter.

The reduced pressure (lower than the injection pressure of the fibrosis-causing agent) in the region beyond the sheath facilitates the introduction and administration of the fibrosis-causing agent at a constant pressure into the region beyond the catheter. No specific restrictions are imposed on the method of controlling the pressure at the fore-end of the sheath or the fore-end of the catheter. To be specific, the pressure control may be accomplished by inserting the catheter into the sheath through a sealing valve attached to the proximal end of the sheath. The sealing valve closes the alveolar parenchyma beyond the fore-end of the sheath. This permits easy pressure control at that part.

It is also possible to control pressure in the alveolar parenchyma beyond the fore-end of the sheath, if the proximal end of the sheath is provided with a three-way stopcock through which air is introduced and discharged. The foregoing method may be applied also to the pressure control beyond the fore-end of the catheter. The sealing valve attached to the base of the catheter closes the alveolar parenchyma beyond the fore-end of the catheter. This permits easy pressure control at that part. It is also possible to control pressure in the alveolar parenchyma beyond the fore-end of the catheter, if the proximal end of the catheter is provided with a three-way stopcock through which air is introduced and discharged. Moreover, the inflation and deflation of the catheter's balloon may be accomplished in any way, without specific restrictions, by means of the three-way stopcock attached to the proximal end of the catheter. In addition, the catheter may have a lumen for a guide wire which facilitates the insertion of the catheter to the desired position.

The catheter suitable for the foregoing method is one which is provided with a balloon to close the bronchus and also with a lumen which has openings at a far part and a near part and delivers a liquid to the far part. Another example of the catheter is a percutaneous transluminal coronary angioplasty (PTCA) catheter of over-the-wire (OTW) type which is designed for treatment of cardiovascular stenosis. These catheters may be any commercial ones listed below. Microcatheter (FINECROSS®, made by Terumo Corp.) that permits passage of a guide wire to cardiovascular stenosis. PTCA catheter (Ryujin Plus OTW® made by Terumo Corp.). Occlusion microballoon catheter (ATTENDANT® made by Terumo Clinical Supply Co., Ltd.). The foregoing catheter is inserted into the bronchus through the working lumen of a bronchoscope. Using a bronchoscope is not essential if the catheter is arranged at any desired position. The catheter and the catheter's balloon (in its inflated state) are not specifically restricted in diameter; an adequate diameter should be selected according to the diameter of the bronchus and bronchiole. To be concrete, the outside diameter of the inflated balloon of the catheter should preferably be slightly larger than the inside diameter of the bronchus or bronchiole in which the fore-end of the inserted catheter lies. To be more specific, the outside diameter (Y mm) of the inflated balloon should be about one to two times larger than the inside diameter (X mm) of the bronchus or bronchiole. This ratio is suitable for the catheter or balloon to come into close contact with the bronchus or bronchiole (which is formed from elastic smooth muscles) without severe damage.

This step (a) may be carried out in such a way that, prior to insertion of the catheter into the bronchus or bronchiole, a guide wider is inserted into the catheter's lumen (for fluid delivery). Manipulation in this way permits the fore-end of the guide wire to be placed beyond the fore-end of the catheter or near the peripheral position. Thus, the fore-end of the catheter can be introduced to the vicinity of pulmonary alveoli or alveolar sacs (air sacs) beyond the bronchus or bronchiole. The guide wire to be used for this purpose may be any known one designed for pulmonology, cardiology, and gastroenterology. It should have an adequate outside diameter which depends on the size of the lumen of the catheter to be used. Its typical example is Runthrough® for cardiology, having an outside diameter of 0.014 inch, made by Terumo Corporation.

It is desirable that the fore-end of the guide wire and catheter be provided with a member (agent) capable of radiographic imaging. This arrangement permits the operator to confirm the position of the fore-end of the guide wire and catheter (which projects from the fore-end of the endoscope) at the time of observation by X-ray radioscopy. In this way the operator can introduce the guide wire and catheter to the respiratory region (including enlarged pulmonary alveoli or alveolar sacs) which have previously been identified by X-ray radioscopy or computed tomography (CT) scan. In this occasion, the guide wire is pulled away after it is confirmed by X-ray radioscopy that the fore-end of the catheter has reached the desired position. The foregoing operation should preferably be performed in such a way that the fore-end of the guide wire is placed beyond the fore-end of the catheter. Moreover, the fore-end of the catheter should preferably have a network structure or perforated structure so that it will not adhere to the inner wall of the respiratory region (such as pulmonary alveoli and alveolar sacs).

Step (b)

This step is intended to administer the fibrosis-causing agent according to the present disclosure to the respiratory region (including pulmonary alveoli and alveolar sacs) through the catheter which has been inserted by the step (a) mentioned above. The operation by this step effectively places the fibrosis-causing agent in the affected part (enlarged alveolar parenchyma), thereby inducing and promoting fibrosis in the affected part and hence reducing the lung capacity.

As mentioned above, the fibrosis-causing agent is heavily relies on its particle size (diameter) so that it stays in the affected part (enlarged alveolar parenchyma) and effectively induces and promotes fibrosis there. Therefore, it is desirable to introduce or administrate the fibrosis-causing agent to the affected part based on the entrance diameter of the affected part in the case of general patients suffering from pulmonary emphysema without actual measurement of the entrance diameter of the affected part. Alternatively, it is desirable to measure the entrance diameter of the affected part (enlarged alveolar parenchyma) prior to the step (b). The latter is more preferable because the entrance diameter of the affected part varies according to the weight and seriousness of the patient and the position of the affected part. In other words, the method of administration to be used for the present disclosure should preferably involve an additional step of measuring the entrance diameter of the enlarged pulmonary alveoli and alveolar sacs prior to the step (b).

There are no specific restrictions on the method for measurement of the entrance diameter of enlarged pulmonary alveoli and alveolar sacs. Any known methods may be employed, such as measurement by means of CT scan or an endoscope, X-ray radiography with the help of a contrast medium delivered into the bronchus, and observation through a probe inserted to the vicinity of the entrance of the pulmonary alveolus and alveolar sac which have been made visible by ultrasound or infrared rays.

The particle diameter of the fibrosis-causing agent should preferably be determined according to the entrance diameter of the affected part which has been measured as mentioned above. In other words, after the entrance diameter of the patient's pulmonary alveolus or alveolar sac is measured, the particle diameter of the fibrosis-causing agent to be administered should be determined prior to the step (b) based on the measured entrance diameter of the affected part. In this way it is possible to administer the fibrosis-causing agent which has a particle diameter suitable for it to effectively stay in the affected part (or enlarged alveolar parenchyma) and induce and promote fibrosis. Thus, the fibrosis-causing agent of the present disclosure securely stays at the affected part and surely induces and promotes fibrosis in the affected part (or atrophy of the affected part), thereby reducing the lung capacity rapidly and certainly. There are no specific restrictions on the relation between the entrance diameter of the affected part (or enlarged alveolar parenchyma) and the particle diameter of the fibrosis-causing agent to be administered. However, it is desirable to satisfy the relation between them as mentioned above. Incidentally, it is acceptable to perform continuously or separately (with an adequate break) the step to determine the entrance diameter of the affected pulmonary alveolus or alveolar sac and the step to determine the particle diameter of the fibrosis-causing agent to be administered. Moreover, these two steps may be executed simultaneously (or continuously) with or prior to the administration of the fibrosis-causing agent.

The amount of administration of the fibrosis-causing agent is not specifically restricted so long as it is sufficient to induce and promote fibrosis in the affected part; it varies depending on the type and weight of the patient, the seriousness of pulmonary emphysema, and the position of insertion of the catheter. An adequate amount of administration to a patient suffering from pulmonary emphysema is 0.1 to 50 mL/kg (of weight), preferably 0.3 to 10 mL/kg (of weight). This dosage will be adequate to satisfactorily induce and promote fibrosis (atrophy) in the affected part (such as enlarged alveolar parenchyma), thereby reducing the lung capacity.

Upon administration as mentioned above, the fibrosis-causing agent of the present disclosure brings about fibrosis (or atrophy) in the inner wall of the enlarged alveolar parenchyma, thereby reducing the lung capacity. Moreover, it maintains the state of fibrosis (atrophy) in the enlarged alveolar parenchyma, thereby reducing the lung capacity. It also maintains the reduced lung capacity during respiration. This results in alleviation and prevention of the lung's overexpansion which weakens the patient due to pulmonary emphysema or bronchus occlusion. Fibrosis induced as mentioned above is so effective as to make the enlarged alveolar parenchyma smaller than its original size; this effect in turn controls and prevents oppression and occlusion of surrounding bronchi by the enlarged alveolar parenchyma. Moreover, the fibrosis-causing agent of the present disclosure is administered through a catheter, without the necessity of surgical operation. This reduces burdens on the patient. Finally, the fibrosis-causing agent of the present disclosure grows connective tissues (particularly fibrocytes) in the inner wall of the enlarged alveolar parenchyma, and hence it recovers the resilience of the enlarged alveolar parenchyma, thereby controlling and preventing the lung's overexpansion.

### EXAMPLES

The present disclosure produces the effect as demonstrated by the following Examples and Comparative Examples, which are not intended to restrict the scope thereof. Experiments in these examples were carried out at room temperature (25° C.), unless otherwise stated.

### Example 1

An aqueous solution (1% w/v) of calcium chloride was prepared by dissolving 6.0 g of calcium chloride (made by Wako Pure Chemical Industries, Ltd.) in 600 mL of reverse osmosis water (RO water). An aqueous solution (1% w/v) of sodium alginate was also prepared separately by dissolving 1.5 g of sodium alginate (made by Wako Pure Chemical Industries, Ltd.) in 150 mL of reverse osmosis water (RO water).

The sodium alginate solution (150 mL) prepared as mentioned above was added (in an atomized state with stirring) to the calcium chloride solution (600 mL) prepared as mentioned above. Thus there was obtained a solution containing calcium alginate in particulate form varying in particle size. This solution was sifted through a sieve having a mesh size of 100 μm, sieves having mesh sizes of 150 μm and 250 μm, and sieves having mesh sizes of 200 μm and 300 μm, so that the particles therein were classified into three groups each having a particle diameter no larger than 100 μm, no smaller than 150 μm and no larger than 250 μm, and no smaller than 200 μm and no larger than 300 μm. The thus separated particles were thoroughly washed with an aqueous solution of calcium chloride and then allowed to stand overnight in it. With the supernatant discarded by using an aspirator, the remaining solution was centrifuged at 500×g for three minutes. The resulting precipitates were washed and sterilized three times with 70% ethanol, and the supernatant was discarded. The remaining particles were suspended in as much distilled water (made by Otsuka Pharmaceutical Co., Ltd.) as half the volume of the particles. The particles in the suspension were examined for average particle diameter by using an LS particle size distribution measuring apparatus (Beckman Coulter). The average particle diameter measured in this manner was as follows.

Batch 1 of particles passing through 100-μm screen: 89 μm

Batch 2 of particles passing through 150-μm and 250-μm screens: 178 μm

Batch 3 of particles passing through 200-μm and 300-μm screens: 262 μm

### Comparative Example 1

An aqueous solution (0.5% w/v) of sodium alginate was prepared from 0.15 g of sodium alginate (made by Wako Pure Chemical Industries, Ltd.) dissolved in 30 mL of RO water. It was sterilized by filtration through Millipore (0.22 μm). The resulting aqueous solution of sodium alginate was a viscous fluid.

### Comparative Example 2

An aqueous solution (40 mM) of calcium chloride was prepared from 0.222 g of calcium chloride dissolved in 50 mL of RO water. It was sterilized by autoclaving at 121° C. for 20 minutes.

An aqueous solution (0.5% w/v) of sodium alginate was also prepared from 0.15 g (made by Wako Pure Chemical Industries, Ltd.) dissolved in 30 mL of RO water. It was sterilized by filtration through Millipore (0.22 μm).

The 0.5% (w/v) aqueous solution of sodium alginate and the 40 mM aqueous solution of calcium chloride were mixed together in a ratio of 2:1 by volume. The resulting mixture was a gel.

Experiments for Evaluation of Fibrosis

The products obtained above are designated as follows.

Sample 1:

The aqueous solution (0.5% w/v) of sodium alginate obtained in Comparative Example 1.

Sample 2:

The gel-like product obtained in Comparative Example 2.

Samples 3 to 5:

The first to third batches of particles obtained in Example 1.

For the purpose of evaluation, each of these samples was administered to the lung tissue of a rabbit in the following manner according to the dosage shown in Table 1.

TABLE 1

| Sam- | Product | | | | |
|---|---|---|---|---|---|
| ple | administered | Dosage | | Form | Remarks |
| 1 | Sodium alginate | 2.0 mL | | Viscous | Comparative |
| | | | | fluid | Example 1 |
| 2 | Sodium alginate | 2.0 mL | 3.0 mL | Gel | Comparative |
| | Calcium chloride | 1.0 mL | | | Example 2 |

| | | | | |
|---|---|---|---|---|
| 3 | Calcium alginate | 2.0 mL | Particles | Example 1, |
| | particles no | | | Batch 1 |
| | larger than 100 μm | | | |
| 4 | Calcium alginate | 2.0 mL | Particles | Example 1, |
| | particles no | | | Batch 2 |
| | smaller than 150 μm | | | |
| | and no | | | |
| | larger than 250 μm | | | |
| 5 | Calcium alginate | 2.0 mL | Particles | Example 1, |
| | particles no | | | Batch 3 |
| | smaller than 200 μm | | | |
| | and no | | | |
| | larger than 300 μm | | | |

1. Method for Surgery

A Japanese white rabbit (clean, male, 3.0 to 4.49 kg) was given (by intramuscular injection) xylazine hydrochloride (diluted four times with physiological saline) at a dose of 5 mg/kg (1 mL/kg) for preanesthetic medication.

An injection drug of anesthetic was prepared from somnopentyl (sodium pentobarbiturate, made by Kyoritsuseiyaku Corp.), which was diluted with 3.24 times as much physiological saline so as to give about 3 mL (3.0 to 3.49 mL) of solution containing as much active ingredient as 20 mg/kg (1 mL/kg). This solution (1 mL) (1.0 to 1.49 mL) was injected into the rabbit through its auricular vein for anesthesia. Incidentally, the remaining solution (2 mL) was additionally administered 0.5 mL each when the rabbit showed reflex during operation so that the rabbit keeps the desired anesthetic depth.

With its sufficient anesthetic depth confirmed, the rabbit had its cervical part dissected at the median part thereof, so that the trachea was exposed. Then, a 0.035-inch guide wire (Radifocus®, made by Terumo Corp.) was inserted into the posterior lobe of the right lung through the dissected trachea, until its fore-end reached the position of the seventh rib (or the upper part of the third branch). The guide wire was passed through the lumen of a 6Fr guiding catheter (Shaperon®, made by Terumo Corp.) processed to about 20 cm in length and coated with lidocaine. The catheter was inserted so that its fore-end reached the seventh rib, and finally the guide wire was pulled out.

By way of the catheter, each of the fibrosis-causing agents shown in Table 1 was infused little by little in coincident with inhalation as shown in Table 2. To be more specific, each of samples 1, 3, 4, and 5 shown in Table 1 was infused four times (0.5 mL each), and subsequently (after infusion of 1 mL) 10 mL of air was infused. In the case of sample 2 shown in Table 1, 0.5 mL of the 40 mM aqueous solution of calcium chloride was infused first and then 1 mL of the 0.5% w/v aqueous solution of sodium alginate was infused. This step was repeated twice, and 10 mL of air was infused after each administration.

TABLE 2

| Agent administered | Method of administration | Remarks |
|---|---|---|
| Sodium alginate | 1 mL (0.5 mL each, four times), | Comparative |
| | followed by infusion of air | Example 1 |
| | (10 mL) | |
| Sodium alginate | Sequential administration of | Comparative |
| plus calcium | calcium chloride (0.5 mL) and | Example 2 |
| chloride | sodium alginate (1 mL), repeated | |
| | twice, with each administration | |
| | followed by infusion of air (10 mL) | |
| Calcium alginate | 1 mL (0.5 mL each, four times), | Example 1 |
| | followed by infusion of air | |
| | (10 mL) | |

After administration of the fibrosis-causing agent, the rabbit had its trachea sutured and then was given a parenteral solution of viccillin antibiotics (0.5 g of ampicillin sodium* diluted with 10 mL of physiological saline) at a dose of 2 mL (100 mg/head) by intramuscular injection at the paradissected part. (* made by Meiji Seika)

2. Autopsy and Histologic Examination

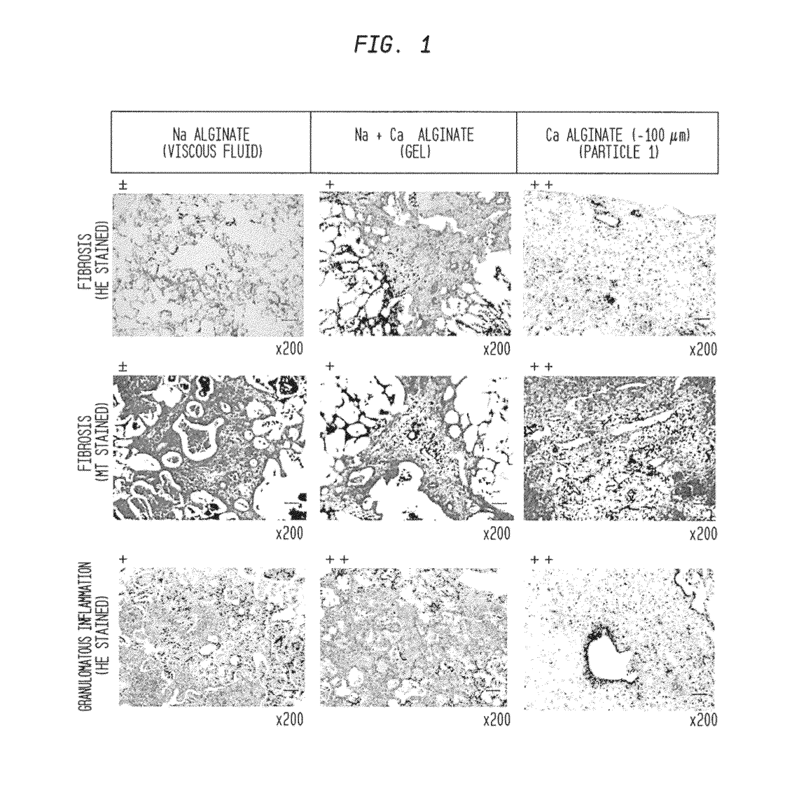

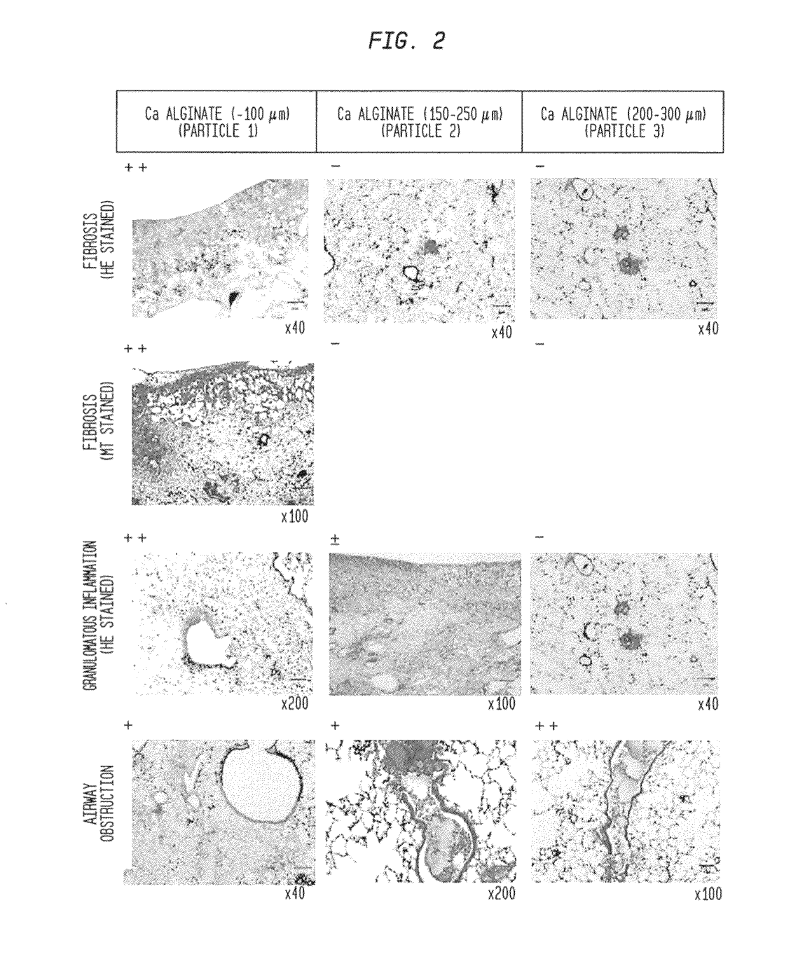

One week or four weeks after the surgery mentioned above in Paragraph 1, the rabbit was given an anesthetic by intravenous injection through its auricular vein. The anesthetic is somnopentyl (sodium pentobarbiturate) diluted twice with physiological saline, so that its dosage is 45 mg/kg (1 mL/kg). The amount of the solution injected was 4.86 mL to 5.65 mL. The rabbit under anesthesia underwent laparotomy in dorsal position. Then, it underwent perfusion through the heart with physiological saline (containing heparin, 10 units/mL, 100 mL/head), so that it was killed by bleeding from the abdominal aorta. Finally, it had its lung extracted. Into the extracted lung was injected (at a water-gauge pressure of 25 cm) 10% buffered formalin as a preserving and fixing solution for pathologic tissues (which contains, in 100 mL, 10 mL of formalin (35.0 to 38.0% aqueous solution of formaldehyde), 0.4 g of sodium dihydrogenphosphate, and 0.65 g of sodium monohydrogenphosphate anhydride, with the rest being purified water). For immersion fixation, the lung was allowed to stand for 24 hours in the 10% buffered formalin. Subsequently, specimens were prepared by paraffin embedding and staining with hematoxyline-eosin (HE stain) and masson trichrome (MT stain). The specimens were pathologically examined under an optical microscope for fibrosis and granulomatous inflammation (which would lead to fibrosis) in lung tissues. The presence or absence of fibrosis and the presence or absence of granulomatous inflammation were judged. Evaluation was made according to the ratings below from the results of observation. FIGS. 1 and 2 show the microphotographs taken one week after administration of the fibrosis-causing agent. Observation after lung extraction showed that the particles of batches 1 to 3 stay as desired in the pulmonary alveolus (or at the spot of administration).

The batches 1 to 3 of particles were evaluated as follows to see if they cause trachea occlusion. The result is shown in the microphotographs (FIG. 2) which were taken one week after administration. The HE-stained image of tissues treated with the batch 1 of particles (calcium alginate, up to 100 μm in diameter) is a magnified version (40 times) of the image (×200) shown in FIG. 1.

Rating of Fibrosis and Granulomatous Inflammation

−: No change (Specimens show no fibrosis and granulomatous inflammation)

±: Very slight (Specimens show fibrosis and granulomatous inflammation at 1 place)

+: Slight (Specimens show fibrosis and granulomatous inflammation at 2 to 4 places)

++: Medium (Specimens show fibrosis and granulomatous inflammation at 5 to 9 places)

+++: Serious (Specimens show fibrosis and granulomatous inflammation at 10 places or more)

Rating of Trachea Occlusion

−: No occlusion

+: Slight (Specimens show trachea occlusion at 2 to 4 places)

++: Medium (Specimens show trachea occlusion at 5 to 9 places)

It is noted from FIG. 1 that batch 1 of the particles induces fibrosis and granulomatous inflammation (which would lead to fibrosis) more significantly than the viscous solution of sodium alginate in Comparative Example 1 and the gel in Comparative Example 2. It is also noted from FIG. 1 that the effect of fibrosis increases in the order of viscous fluid, gel, and particles. A probable reason for this is that particles stay at the point of administration more easily than fluid and gel and particles secure a sufficiently large area in contact with alveolar tissues (which leads to active reactions with living bodies).

It is noted from FIG. 2 that batch 1 of the particles brings about fibrosis more significantly (without airway occlusion) than the batches 2 and 3 of particles. This is due to the fact that the batch 1 of particles in a sufficient amount smoothly passes through the bronchus and reaches the pulmonary alveolus, thereby securing a sufficient area in contact with alveolar tissues. By contrast, the batches 2 and 3 of particles (with a larger particle diameter) partly get caught in the bronchus and reach the pulmonary alveolus in too small a quantity (compared with the batch 1 of particles) to bring about fibrosis. Incidentally, the rabbit used in these examples normally has an entrance diameter of pulmonary alveoli or alveolar sacs which ranges from 100 to 160 μm. However, the enlarged pulmonary alveoli or alveolar sacs of human patient have an entrance diameter of 1 to 2 mm. This means that particles smaller than 2 mm can enter the pulmonary alveoli and bring about fibrosis there. In addition, the catheter inserted to the vicinity of the entrance of the pulmonary alveoli or alveolar sacs make it possible to push in those particles having a particle diameter nearly twice the entrance diameter of the pulmonary alveoli or alveolar sacs. Therefore, it is conjectured from FIG. 2 that the batches 2 and 3 of particles bring about sufficient fibrosis and stay long in pulmonary alveoli when administered to a human patient, and hence they effectively induce fibrosis.

It is concluded from the foregoing that the particles capable of inducing fibrosis should have a particle size that permits their entry into pulmonary alveoli and secures a sufficient area of their contact with alveolar tissues.

It should be understood by those skilled in the art that various modifications, combinations, sub-combinations and alterations may occur depending on design requirements and other factors insofar as they are within the scope of the appended claims or the equivalents thereof.

What is claimed is:

1. A fibrosis-causing agent in a particulate form.

2. The fibrosis-causing agent according to claim 1, which is used for therapy of pulmonary emphysema.

3. The fibrosis-causing agent according claim 2, wherein the particles have a diameter no larger than 1× the entrance diameter of enlarged pulmonary alveoli or alveolar sacs.

4. The fibrosis-causing agent according to claim 2, wherein the particles have a diameter larger than an entrance diameter of normal pulmonary alveoli or alveolar sacs.

5. The fibrosis-causing agent according to claim 1, which contains at least one compound selected from the group consisting of alginate and alginic ester.

6. The fibrosis agent according to claim 1, wherein said particulate form comprises a fibrosis promoter and/or a fibrosis inducer.

7. The fibrosis-causing agent according to claim 1, wherein the particulate form remains at or on an affected part to promote and/or induce fibrosis

8. The fibrosis-causing agent according to claim 7, wherein the affected part is pulmonary alveoli or alveolar sac.

9. The fibrosis-causing agent according to claim 7, wherein the particulate form does not flow out of the affected part.

10. The fibrosis-causing agent according to claim 4, wherein the particulate form has a diameter in the range from approximately 2000 μm to 100 μm.
